(19) United States
(12) Patent Application Publication
(10) Pub. No.: US 2006/0189560 A1
(43) Pub. Date: Aug. 24, 2006

(54) Molecular method to augment RNA mediated gene silencing

(76) Inventor: Ajit Kumar, Bethesda, MD (US)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/340,561

(22) Filed: Jan. 27, 2006

Related U.S. Application Data
(60) Provisional application No. 60/646,992, filed on Jan. 27, 2005.

(51) Int. Cl. A61K 48/00; C12N 15/87

(52) U.S. Cl. 514/44; 435/455

(57) ABSTRACT

The invention provides for a novel method of augmenting gene silencing via RNA interference (RNAi). Under the invention, RNAi technology is combined with the action of a variant nuclear factor to potently inhibit gene expression. In one embodiment of the invention, the nuclear factor is a variant of a double-stranded RNA (dsRNA) binding protein termed NF90ctv. The invention is also related to diagnostic/investigative and treatment methods and to cell lines produced by the methods disclosed.

### RELATED APPLICATION

This application claims priority of U.S. provisional application, application No. 60/646,992 filed Jan. 27, 2005, the disclosure of which is hereby incorporated by reference.

### GOVERNMENT INTEREST

This invention was made with U.S. government support under National Institutes of Health grant number AI 054222. The U.S. government may have certain rights in the invention.

### BACKGROUND OF THE INVENTION

1. Field of the Invention

The present invention relates to the field of gene silencing through the use of RNA interference (RNAi) technology. More particularly, the invention relates to methods for the enhancement of RNAi-mediated gene silencing using short interfering RNAs (siRNA) in conjuction with one or more enhancing agents.

2. Description of the Related Art

The regulation of gene expression occurs at many levels, broadly categorized as pre-transcriptional, transcriptional and post-transcriptional control mechanisms, depending upon where in the expression pathway the control is exerted.

As the name implies, post-transcriptional control of gene expression occurs after the DNA template has been transcribed into mRNA. The level of control has been historically characterized by alternative splicing of the mRNA, mRNA and protein decay rate, and the like, as well as regulation of translation. More recently, a new level of post-transcriptional control has been recognized. This mechanism of control of gene expression is mediated by small interfering RNA (siRNA).

Small Interfering RNA (siRNA) and RNA Inhibition (RNAi).

In 1998, Fire et al. first described a novel mechanism for post-transcriptional gene regulation. Specifically, Fire et al. demonstrated that small RNA molecules are able to specifically down-regulate the level of gene expression in the nematode Caenorhabditis elegans. ((Fire, A., et al., Nature, 391, 806-811 (1998)). See also, U.S. Pat. No. 6,506,559. These authors demonstrated that this gene-specific “silencing” is mediated by short segments of double-stranded RNA (dsRNA) that are homologous to the messenger RNA (mRNA), or precursors thereof, of the gene of interest. These silencing RNAs have be termed “small-interfering RNA” (siRNA), and methodology employing their use is termed “RNA interference” (RNAi).

Naturally occurring small interfering RNAs (siRNAs) are double-stranded RNAs, generally about 20 to 30 nucleotides in length. The siRNAs participate in the specific ablation of cellular RNAs, thereby preventing their expression within the cell. The siRNAs have a 5′-phosphate/3′-hydroxyl structure, and may have a short overhang on the 3′ end of each strand. (Caplen, et al., Proc. Natl. Acad. Sci. U.S.A., 98(17) 9742-9747 (2001)).

Synthetic siRNAs are also generally of a length of about 20 to 30 nucleotides. These RNAs may be synthesized in vitro using well-characterized transcription systems or chemical synthesis techniques, and introduced into a cell of interest either as two independent and substantially complementary single-stranded RNAs (ssRNA) or as hybridized dsRNA. Additionally, a single, self-hybridizing ssRNA molecule having internal complementary sequences and capable of forming a stem-loop structure can also be used. Small interfering RNAs may also be produced in vivo though the use of appropriately designed expression vectors, including recombinant viruses, plasmids, and the like, using conventional means.

Small interfering RNAs are thought to exert their effect through their incorporation into a ribonucleoprotein (RNP) complex that specifically degrades targeted RNA species; the siRNA in this RNP complex provides the specificity of targeting via the Watson-Crick base pairing of one strand of the siRNA with complementary bases on targeted RNA species.

Small interfering RNAs are evolutionarily ancient, as cellular mechanisms for producing these RNAs, as well as the cellular responses to them, are found in plants, animals, and a variety of protozoa. Depending upon the particular organism, the siRNAs provide a cellular defense against viral infection, or expression of endogenous viral or transposable element sequences.

In vivo in eukaryotes, siRNAs are generated by the relatively non-specific cleavage of longer dsRNA species into dsRNAs approximately 20 to 30 nucleotides in length. An endoribonucleolytic “Dicer-like” activity is responsible for this cleavage. Once these short dsRNA species are generated, one of the strands enters the RNA-induced silencing complex (RISC), which is able to cleave and/or inhibit translation of the targeted RNA species. Gene silencing is thus achieved by the ablation and/or translational inhibition of the targeted mRNA. Additionally, in certain organisms this degradation of the target RNA may occur in conjunction with, or through the action of, a dsRNA-dependent RNA polymerase activity, the net result being an amplification of the number of siRNA molecules (and concomitant effective increase in gene silencing therethrough).

RNAi methodology can be highly gene specific. However, the siRNA-mediated RNAi response is somewhat short lived (at best two to three days), and thus of somewhat limited utility. Therefore, improvement in the duration of the effectiveness of RNAi-mediated gene silencing is highly desirable.

Enhancing Agents.

There are heretofore no known enhancing agents for use in RNAi technology. As meant herein, enhancing agents are those molecules that when used in conjunction with siRNAs enhance the half-life and/or efficiency of the RNA inhibition of gene expression as compared to siRNA alone.

Nuclear Factor 90 (NF90).

Nuclear Factor 90 (NF90) is a multi-functional protein found in a wide variety of organisms. Sequence analysis has shown that NF90 is highly related to the mitotic phosphoprotein MPP4 (also known as the interleukin enhancer binding factor 3 (ILF3) transcriptional activator). Indeed, NF90 and MPP4/ILF3 have been proposed to be splice variants of a single gene located on human chromosome 19 (Duchange, et al., Gene 261: 345-353 (2000)). Further analysis of both NF90 and MPP4/ILF3 indicated that both these proteins carry two copies of a conserved dsRNA binding motif. Subsequently, NF90 was found to efficiently bind to dsRNA and may possibly play a role in a cellular dsRNA-binding protein complex.

In addition to binding dsRNA (and other highly structured RNAs), NF90 has also been shown to interact with the dsRNA-activated protein kinase (PKR), which participates in the interferon-mediated antiviral cascade. Moreover, NF90 has been shown to be phosphorylated in the RNA binding domain by PKR, although the significance of this modification is not yet known. (Parker, et al., J. Biol. Chem., 276(35): 32522-30, (2001)).

NF90 has also been reported to interact with methyltransferase 1 (PRMT1).

Still another of the various roles of NF90 is the sequence specific binding in the 3′ untranslated region (3′ UTR) of the interleukin-2 (IL-2) mRNA. This binding is believed to stabilize the IL-2 mRNA in activated T-cells. Indeed, it has been shown that NF90 is localized to the nucleus in quiescent T-cells, and upon T-cell activation is at least partly exported to the cytoplasmic compartment, and that this nuclear export is required for IL-2 mRNA stabilization.

Yet another of the many roles of NF90 in gene regulation is that of the inhibition of translation and alteration of mRNA binding to polyribosomes. An example of such inhibition is that of the acid beta-glucosidase gene. (Xu, et al., Mol. Genet. Metab., 70(2): 106-115 (2000)).

A distinct protein also designated NF90 has also been described in the literature. This protein is a subunit of the Nuclear Factor of Activated T-Cells (NFAT) transcription factor. However, it should be noted that molecular studies have clearly demonstrated that the NF90ctv, as described in the present invention, is unrelated to the NF90 protein associated with NFAT.

Finally and importantly, Krasnoselskaya-Riz, et al., have reported a C-terminal variant of the NF90 protein (NF90ctv) (coding sequence accession number U10324) (Krasnoselskaya-Riz, et al, AIDS Res. Hum. Retroviruses, 18(8):591-604 (2002); incorporated herein by reference). This variant is approximately 670 amino acids in length, and is the result of a CT insertion in exon 15 of the NF90/MPP4/ILF3 gene. As a consequence of this dinucleotide insertion, the amino acid sequence downstream is in an altered reading frame relative to the wild-type protein. This altered reading frame results in substitution of an arginine/glycine rich region with a largely acidic region of approximately 69 amino acids at the C-terminus.

Krasnoselskaya-Riz, et al., further demonstrated that NF90ctv can activate interferon response genes in the absence of viral infection. It is thought that this interferon response elicited by NF90ctv is mediated, at least in part, via the activation of transcription factors in the interferon response cascade pathway. This study thus demonstrates that NF90ctv may be involved in the transcriptional activation of certain genes. The study did not, however, suggest any involvement of NF90ctv in gene silencing via the RNAi-mediated mechanism.

### SUMMARY

While RNAi technology has proven useful, siRNA alone typically achieves less than a 20% of the level of desired target-specific gene knock down. Additionally, even with the optimal dose, siRNA is typically effective for less than 4 days. This limited usefulness is thought to be due to (i) the short lived siRNA “trigger”, and/or (ii) an inefficient targeting/ablation of the desired mRNA species.

Thus, although RNAi technology has been advantageously employed for various purposes in a wide variety of organisms, cells and/or tissue types, there remains a need in the art for improvement in the efficiency and specificity of gene silencing using the RNAi technique. It would thus be advantageous to have improved methods for enhancing the half-life of the siRNA molecule and/or the efficiency of the targeting and ablation of the desired gene-specific RNA.

The present invention provides for such an enhancement in the technology through the use of novel combinations of siRNA in conjunction with one or more enhancing agents, as well as various associated methods that advantageously make use of these novel combinations The siRNA-mediated gene silencing methods of the invention thus provide novel therapeutics against infectious diseases and cancer by means of a heightened RNAi response. This heightened response is achieved though the combination of siRNA and one or more enhancing agents, most preferably NF90ctv (or derivatives thereof). The present inventors have found that the combination of the invention greatly diminishes the previous limitations of RNAi methodology, leading to up to 100-fold or more enhancement in RNAi-mediated gene silencing over previous methods.

### BRIEF DESCRIPTION OF THE DRAWINGS

No drawings accompany this application.

### SUMMARY OF THE INVENTION

The invention provides for improved methods in the field of RNAi technology, which employ novel combinations of siRNA and one or more enhancing agents. The enhancing agents under the invention provide for improved half-life, effectiveness and/or efficiency in RNAi methodology. In a preferred embodiment, the enhancing agent is one or more carboxy terminal variants of the nuclear factor 90 protein, described herein as NF90ctv.

The invention also provides for primary cells and cell lines containing the novel combination of siRNA and one or more enhancing agents. The primary cells and cell lines under the invention are characterized by an improved half-life and/or efficiency in response to siRNA techniques.

The invention further provides for diagnostic/investigative and treatment methods for host cells and multicellular organisms. Preferably, the multicellular organism is an animal, more preferably a primate, and most preferably a human. The multicellular organisms treated under the invention are characterized by improved RNAi response resulting from the novel compositions and methodologies described herein.

The invention yet further provides a pharmaceutical composition comprising an siRNA, one or more enhancing agents, and a pharmaceutically acceptable carrier. The enhancing agent under the invention preferably includes the NF90ctv protein or suitable derivatives (including peptide and mutational derivatives) thereof.

### DETAILED DESCRIPTION OF THE INVENTION

The invention we seek to patent concerns the inherent properties of human (and other) cells to control gene silencing. This has been brought into practice by the use of siRNA (small interfering RNA) or mRNA (micro RNA), that seek out complementary sequences within a target mRNA (messenger RNA) and ‘silence’ gene expression by degrading or blocking translation of the mRNA into protein. This ‘onsite’ gene silencing, called RNA interference (RNAi), has important practical advantage since it is designed to eliminate the desired gene product (the mRNA and the protein) without affecting the gene (DNA).

Since the molecular cue for the siRNA-mediated gene knock down is the cell's ability to recognize structural elements of the double-stranded siRNA, we reasoned that ectopic expression of the dsRNA binding protein NF90 should augment dsRNA targeting. We have found this is indeed strikingly the case for the NF90ctv protein.

Exemplary Methods

Design of siRNA Oligonucleotides:

The coding sequence of the mRNA to be targeted for knockdown is scanned, either by sight or by computer, preferably by computer, for an appropriate siRNA complementary sequence. Preferably, the appropriate siRNA sequence lies between about 100 nucleotides from the transcriptional start and similar distance from the translation termination site.

Optimal siRNA is selected from the sequence of complementary siRNAs available (from the sight or computer scan) based preferably on the criteria that the 5′ end of each siRNA sequence is flanked by two or more adjacent adenine residues, and the 3′ end is flanked by two more uridine residues (thymidine residues in the DNA). It is also preferred that the siRNA sequence not contain a substantially contiguous internal stretch of about four or more adenine or uridine residues.

To confirm that the selected siRNA specifically knocks down the targeted mRNA, (i.e., does not “off-target” non-specific or other specific mRNAs), a BLAST is preferably performed to identify possible homology between the proposed siRNA and other coding sequences.

Selected siRNAs with the fewest BLAST matches are preferably chemically synthesized. These siRNAs may be obtained through a variety of commercial suppliers or, alternatively, may be synthesized via established chemical methods as known to those skilled in the art.

For use under the invention, siRNAs may also be preferably obtained via recombinant DNA methodologies. For cloning into an expression vector, one preferred embodiment for generating siRNAs employs oligonucleotides of, for example, the following general structure:

```
Forward Strand
GATCCCCXXXXXXXXXXTTCAAGAGAXXXXXXXXXXTTTTTGGAA
                  sense orientation anti-sense
Reverse Strand
AGCTTTCCAAAAAXXXXXXXXXXTCTCTTGAAXXXXXXXXXXGGG
                                sense orientation anti-sense
```

The siRNA-encoding DNA sequence (here, a 29 nucleotide sequence in proper orientation bounded by the bolded and underlined areas above) is selected so as to specifically encode a siRNA directed to the target gene. When hybridized, such an oligonucleotide pair can be conveniently directionally ligated into restriction endonuclease sites of a suitably designed and cleaved (e.g., BamHI and HindIII) expression vector.

These synthetic DNA oligonucleotides are annealed, phosphorylated if desired, and cloned into a suitable expression vector, such as the commercially available H1 promoter-containing vector, pSUPER (Brummelkamp, et al., Science 19:296(5567):550-3 (2002); OligoEngine, Inc., Seattle, Wash.). The plasmid DNA expression vector containing the siRNA-encoding insert is preferably amplified in bacteria and purified using standard protocols.

Enhancing Agents:

A preferred enhancing agent under the invention is a “carboxy terminal variant” of the NF90 protein, referred to herein as NF90ctv. NF90ctv is approximately 670 amino acids in length. The carboxy terminal end of the NF90 protein has an insertion of 2 base pairs that results in a frameshift mutation. The mutant is apparently the result of a post-transcriptional insertion of a CT dinucleotide to create the frame shift. The frame shift in NF90ctv turns what is natively an Arg/Gly rich region in the MPP4/IL3 sequence into an approximately 69 amino acid C-terminal end containing Glu/Gln residues. The mutant acts as a negative dominant with respect to the endogenous protein.

NF90ctv contains a potent nuclear localization signal, and is indeed localized to the nuclear compartment. This nuclear localization is thought to be required for the enhancing effect on RNAi-mediated gene silencing under the invention. However, NF90ctv may also act as a shuttle in some situations.

As described above, NF90ctv is related to MPP4-90/(ILF-3). However, the endogenous NF90/MPP4/ILF3 does not appear to operate in augmenting the RNAi response under the invention.

Transfection of Mammalian Cells:

Recombinant plasmids expressing NF90ctv and the target specific siRNA vector are co-introduced into, for example, human cells by cationic liposome-mediated transfection. The heightened response of gene specific silencing is observed (by immunoblotting and loss of function assays) within twenty-four hours of administering NF90ctv. For greater, sustained RNAi response it is preferred that NF90ctv vector be introduced about 24 hours prior the target-specific siRNA.

It will also be appreciated that the siRNA and/or the NF90ctv need not be introduced into the cells in the form of a vector. Indeed, siRNA may be introduced as short 20-30 nucleotide RNAs or as longer, self-hybridizing RNAs as described above. Additionally, the NF90ctv protein may be produced, for example in E. coli or yeast cells, isolated via known methods, and introduced into the target cells in a proteinaceous form.

It should still further be appreciated that the exact amino acid sequence of the NF90ctv protein exemplified herein, while preferred, need not be used in order to practice the invention. For example, an NF90ctv protein expressing less than the entire C-terminal variant end, or one lacking a full complement of the C-terminal acidic amino acids, is expected to be operable under the invention. Likewise, the invention contemplates the use of peptides derived from the NF90ctv protein that are effective in enhancing the siRNA response.

Generally, approximately 5×10⁴ cells at about 60% confluence are transfected (in serum-free, and antibiotic free RPMI culture medium) by lipofection using FuGENE6 (H. Roche, using the manufacturer's conditions), with 1-2 μg target specific siRNA or the siRNA vector (the conditions being optimized for each gene-specific knock down). After at least about 48 hours the cell lysate is assayed for the extent of protein depletion by Western blot, utilizing specific monoclonal or polyclonal sera. In conjunction (or alternatively), a loss of function assay can be used to determine the extent of the inhibition of gene-specific expression.

Transfection and/or of siRNA expression may also be accomplished by other means, such as electroporation, or DEAE-mediated or calcium phosphate-mediated co-precipitation, as known in the art.

When used in the practice of the invention, the siRNA expression vectors may be integrated or extrachromosomal. These vectors may be plasmid vectors, viral vectors (for example, retroviral vectors, adenoviral vectors and AAV vectors), and the like. Expression from the vectors may be either constitutive or regulated.

### Preferred Embodiments

For a 3:1 Lipid:siRNA Transfection Mixture:

Three microliters lipid (Fugene 6) is added to 97 microliters of serum-free, antibiotic-free cell culture medium (for a total volume of 100 microliters). After an approximately 5 minute incubation at room temperature, 1 microgram of siRNA is added. Following an additional incubation of at least 15 minutes at room temperature, the mixture is added dropwise into the well for the transfection.

For a 3:2 Lipid:siRNA Transfection Mixture:

Three microliters lipid (Fugene 6) is added to 97 microliters of cell culture medium containing no serum or antibiotics (for a total volume of 100 microliters). After an about 5 minute incubation at room temperature, 2 micrograms of siRNA is added. Following further incubation for at least about 15 minutes at room temperature, the mixture is added dropwise into the well for the transfection.

For a 6:1 Lipid:siRNA Transfection Mixture:

Six microliters lipid (Fugene 6) is added to 94 microliters of serum-free, antibiotic-free cell culture medium (for a total volume of 100 microliters). After incubation for 5 minutes at room temperature, 1 microgram of siRNA is added. Following an additional incubation of at least 15 minutes at room temperature, the mixture is added dropwise into the well for the transfection.

The day before transfection, mammalian cells (for example, human osteosarcoma HOS cells, human T-lymphocytes CEM cells, and human myeloid cell line Om10.1), were seeded at approximately 50,000 cells per 12 mm culture well in 2 ml cell culture media. Seeding 50,000 cells preferably results in a desired cell density of 50-80% confluence the following day. The target mammalian cells were transfected, for example, with cationic lipids following the manufacturer's (Fugene 6; Roche) recommended protocol or by electroporation (as appropriate for optimal transfection efficiency). The transfection conditions are preferably optimized with lipid:siRNA ratios ranging between about 3:1, 3:2, and 6:1, (in a total volume of 100 μl of serum-free, antibiotic-free cell culture medium).

Diagnostic Methods, Investigative Methods and Kits.

The invention also includes diagnostic methods and kits. Generally, such diagnostic methods involve administering an effective amount of one or more siRNAs and one or more enhancing agents to cells, and thereafter determining a characteristic change in said cells. Preferably, the enhancing agent comprises NF90ctv and/or an siRNA-enhancing derivative thereof such as peptide or molecular variant. The siRNA may as designed, manufactured, and administered as above. The enhancing agent, preferably NF90ctv, may be administered as a proteinaceous material or through use of a suitable expression vector, also as described above.

At a suitable time after administration, the expression of the targeted RNA(s) is determined, and correlated with cellular changes induced by the ablation of the target RNA(s). Such methods may be used to diagnose and/or investigate specific cellular conditions that are affected by the enhanced ablation of the target RNA(s). The information so provided is useful for, inter alia, the design of a highly specific therapeutic treatment regimen for a recipient host.

For example, infectious agents and pathogens (e.g., viral and bacterial pathogens) are continually emerging and evolving. As such, specific treatments that were effective in the past may not be effective in treating the newly emerged agent. The diagnostic methods under the invention allow for rapid design and evaluation of new therapeutic strategies using siRNA specific for the agent and, an enhancing agent such as NF90ctv, for effective treatment.

The invention further comprises diagnostic kits containing one or more siRNA and an enhancing agent (such as NF90ctv or a derivative thereof) suitably packaged for storage and/or transport. The siRNA and enhancing agent may be packaged individually or in combined form. Additional reagents, such as transfection materials (e.g., cationic lipids suitable for lipofection), as well as devices for dispensing and/or administration of the reagents may also be included in the kits of the invention. Packaging materials, for example instructions for use of the kit, are additionally included if desired.

Therapeutic Methods and Kits.

The invention further provides for therapeutic methods and kits suitable for use in practicing the invention on host organisms or cells. Generally, these methods comprise administering a therapeutically effective amount one or more siRNAs and an enhancing agent (preferably NF90ctv or a derivative thereof) to a host organism/cells in need thereof for treatment of an abnormal cellular condition, in a pharmaceutically acceptable carrier. Such cellular conditions include pathogenic infections (viral, bacterial, etc.) and cancers.

The host cells may be derived from the host organism or patient and manipulated under the method of the invention, followed by re-insertion into the host organism. An example of such a method is the removal of infected or cancerous cells from a host organism, followed by administration of an siRNA and enhancement agent to the cells and subsequent re-introduction of the cells into the host.

In another treatment methodology under the invention, one or more siRNAs and an enhancing agent such as NF90ctv (or a derivative thereof) may be administered directly to a host organism. Such administration may be, for example, orally, nasally, venously, arterially, intramuscularly, intraperitoneally, and the like. The siRNA and/or enhancing agent may be administered to the host organism in various forms, including aqueous, tablet, capsule, suppository, etc., as known in the therapeutic arts.

Kits for these therapeutic methods are also included in the invention. Such kits are similar to those described above. It is preferred that the kits comprise therapeutically effective amounts of siRNA and enhancing agent. These effective amounts may be packaged in single and/or multiple dose units. It is further preferred that the component/drugs to be administered to the host organism be in substantially sterile form. Administration devices, such as syringes, needles, etc., sterile containers such as small mixing tubes, and packaging material may also be included in the therapeutic kits of the invention, as desired.

Pharmaceutical Compositions

The invention also provides a pharmaceutical composition comprising a therapeutically effective amount of one or more siRNA, one or more enhancing agents, and a pharmaceutically acceptable carrier. Another pharmaceutical composition under the invention comprises a therapeutically effective amount of an siRNA expression vector, an enhancing agent, and a pharmaceutically acceptable carrier. The enhancing agent under this aspect of invention preferably includes the NF90ctv protein or suitable derivatives (including peptide and mutational derivatives) thereof.

Hosts and Cell Types.

The practice of the invention is not meant to be limited to specific hosts, and generally includes both animal and plant hosts. However, vertebrate animal hosts are preferred, most preferably human, under the practice of the invention. Examples of vertebrate animals include fish, mammal, cattle, goat, pig, sheep, rodent, hamster, mouse, rat, primate, and human.

The practice of the invention is also not meant to be limited to specific cell types. Generally, the cells having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Exemplary Advantages of the Invention.

The invention enhances RNAi-mediated gene silencing by hundred fold or more, following the introduction of a genetic variant of dsRNA binding protein, NF90ctv. Thus, investigators now utilizing RNAi for drug discovery for cancer and virus resistance will be able to enhance the effectiveness of their small RNA triggers (siRNA) employing this novel molecular method. Moreover, the enhancement under the invention will significantly affect research and development by reducing the cost of this powerful technology.

The heightened response achieved through RNAi in conjunction with NF90ctv is considerably more stable than that achieve with RNAi alone. The invention thus provides for a next generation of NF90ctv delivery system that allows controlled induction (pulses) of NF90ctv expression to sustain the heightened gene-specific, potent and long lasting therapeutics.

In cases where gene therapy/gene delivery approach is being contemplated for a specific disease, and where gene knock down via RNAi technology may benefit the patient, utilizing an enhancing supplement, such as NF90ctv, augments the benefits by several fold and prolongs the beneficial effect of treatment.

In each case, the delivery of NF90ctv either as a expression vector or as an effector peptide against a particular disease target or the pathogen markedly enhances the benefits of the directed RNAi based treatment. The enhancement substantially decreases the diagnostic and therapeutic costs associated with RNAi technology.

It is understood that various other modifications will be apparent to and can readily be made by those skilled in the art without departing from the scope and spirit of this invention. Accordingly, it is not intended that the scope of the claims appended hereto be limited to the description set forth above, but rather the claims be construed as encompassing all of the features of patentable novelty which reside in the present invention, including all features which would be treated as equivalents thereof by those skilled in the art to which the invention pertains.

I claim:

1. A method for augmenting the silencing of a target gene in a cell comprising:
Providing a short interfering RNA (siRNA) specific for said target gene, and an enhancing agent;
Introducing said siRNA into said cell; and
Introducing an effective amount of said enhancing agent into said cell.

2. The method according to claim 1, wherein said enhancing agent comprises NF90ctv.

3. A method for augmenting the silencing of a target gene in a cell comprising:
Providing a short interfering RNA (siRNA) specific for said target gene, and an expression vector encoding an enhancing agent;
Introducing said siRNA and said vector into said cell.

4. The method according to claim 3, wherein said enhancing agent comprises NF90ctv.

5. A kit for augmenting the silencing of a target gene in a cell comprising;
A short interfering RNA (siRNA) specific for said target gene; and,
An enhancing agent.

6. The kit according to claim 5, wherein said enhancing agent comprises NF90ctv.


SEQUENCE LISTING (2 sequences)

SEQ ID NO: 1 - DNA, 45 bp, Artificial Sequence
  Description of Artificial Sequence Synthetic oligonucleotide
gatccccnnnnnnnnnnttcaagagannnnnnnnnntttttggaa

SEQ ID NO: 2 - DNA, 45 bp, Artificial Sequence
  Description of Artificial Sequence Synthetic oligonucleotide
agctttccaaaaannnnnnnnnntctcttgaannnnnnnnnnggg